(19) United States
(12) Patent Application Publication
(10) Pub. No.: US 2025/0000478 A1
(43) Pub. Date: Jan. 2, 2025

(54) VETERINARY ACOUSTIC AND BIOFEEDBACK DEVICE

(71) Applicant: Quadralynx, Inc., Spokane, WA (US)

(72) Inventors: Mark VanDam, Spokane, WA (US); Gavin La Rowe, Spokane, WA (US)

(21) Appl. No.: 18/754,955

(22) Filed: Jun. 26, 2024

Related U.S. Application Data
(60) Provisional application No. 63/524,663, filed on Jul. 1, 2023.

(51) Int. Cl. A61B 7/02; A61B 5/00

(52) U.S. Cl. CPC A61B 7/02; A61B 5/6802; A61B 5/7264; A61B 2503/40

(57) ABSTRACT

A device includes one or more sensors and one or more output components. The device receives, from the one or more sensors, data, and determines that the data is indicative of sound generated from an animal. Based at least in part on the data being indicative the sound generated from the animal, the device determines one or more biomarkers associated with the sound. The one or more biomarkers include at least an amplitude associated with the sound. The device determines that the one or more biomarkers fail to satisfy a threshold associated with a pathological state of the animal. The device then causes, based at least in part on the one or more biomarkers failing to satisfy the threshold, output of a notification via the one or more output components.

### CROSS-REFERENCE TO RELATED APPLICATION

This application claims priority to U.S. Provisional Patent Application No. 63/524,663, filed Jul. 1, 2023, entitled “Veterinary Acoustic Smart-Detector and Biofeedback Devices and Methods for Detecting and Analyzing Behavioral Activity Signals, for Monitoring Animal Welfare, Diagnosing, Reporting, and Remedying Animal Disease and Distress, and Cueing Animal Behavior to Improve Livestock, Wildlife, and Pet Management and Care,” the entirety of which is herein incorporated by reference.

### BACKGROUND

Monitoring animal behavior and determining animal welfare is often labor-intensive and time-consuming. While household animals, such as birds, cats, or dogs, are often close in proximity to examine, managing livestock and wildlife in pasture and field environments presents a greater challenge. Attempts have been made to develop devices that detect various physical, physiological, and behavioral changes related to animal welfare and management, such as feeding, step count, lameness, milking, etc. However, these devices are often inadequate and fail to provide a complete diagnostic of animal welfare.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features. The systems depicted in the accompanying figures are not to scale and components within the figures may be depicted not to scale with each other.

FIG. 1 illustrates an example device for monitoring animal welfare to determine one or more pathological states of the animal, according to examples of the present disclosure.

FIG. 2 illustrates an example process for monitoring animal welfare to determine one or more pathological states of the animal, according to examples of the present disclosure.

### DETAILED DESCRIPTION

This application is directed, at least in part, to monitoring animal welfare for determining one or more pathological, physiological, anatomical, behavioral, physical, or locative states of the animal, according to examples of the present disclosure. In an embodiment, sensor data generated from one or more sensors may be used to determine animal welfare for detecting behavioral and/or pathological states, such as animal disease, distress, etc. to provide care, treatment, and/or management. For example, the sensor data may be used to determine laryngeal/vocal biomarkers, physical biomarkers, environmental biomarkers, and/or other audio markers associated with the animal or an environment of the animal. The markers may be used to determine the welfare of the animal for deducing pathological states of the animal, for example, by comparing the biomarkers to one or more reference markers. The animal welfare, behavioral, and/or pathological states may be determined in real-time and/or over a period of time (e.g., day, month, year, etc.). In an embodiment, based at least in part on the welfare, behavioral, and/or the pathological states, a caregiver, owner, etc. associated with the animal may be notified to provide care, treatment, etc.

In an embodiment, the techniques described herein may be implemented within, or at least in part, by a device. In an embodiment, the device may be considered a veterinary acoustic and biofeedback device. For example, as will be discussed herein, the veterinary acoustic and biofeedback device may, in an embodiment, generate or utilize sensor data associated with the acoustics of the animal, animal behavior, physiological state, or an environment of the animal to provide feedback to the animal, a caregiver (e.g., owner, veterinarian, etc.) of the animal, and so forth. However, as will be discussed herein, although mentioned as an acoustic device, the veterinary acoustic and biofeedback device may generate or utilize other non-acoustic data (e.g., including biological assessment data, visual data, behavioral data, geolocation, environmental data, etc.) associated with the animal to determine the welfare, behavioral, and pathological states.

The veterinary acoustic and biofeedback device may be a compact, wearable, or peripheral device. For example, the veterinary acoustic and biofeedback device may be worn, attached, mounted, etc., to the animal. In an embodiment, the veterinary acoustic and biofeedback device may be small, non-invasive, non-intrusive, lightweight, and discreet in nature. In an embodiment, the veterinary acoustic and biofeedback device may be embodied within a harness, collar, stick-on arrays, saddle, tag, etc., that is worn by the animal. Additionally, or alternatively, the veterinary acoustic and biofeedback device may be attached or worn by the animal using attachment mechanisms, such as hook and loop, clasps, magnets, cords, chains, clips, belts and buckles, cords, pins, adhesives, etc. for coupling (e.g., pinning, hanging, etc.) in any manner to the animal. Alternatively, the veterinary acoustic and biofeedback device may be sub-dermally implanted, injected, or ingested.

The veterinary acoustic and biofeedback device, however, may also and/or additionally be separate from the animal (i.e., not worn by the animal), such as placed in proximity to the animal. For example, animals may be housed in barns, stables, beds, pens, cages, etc. Here, the veterinary acoustic and biofeedback device (or a component thereof) may be positioned relative to the animal or group of animals or a group of animals to monitor the animal and/or the environment of the animal. In an embodiment, the veterinary acoustic and biofeedback device may be mounted on a wall or door of a pen, for example, attached to a fixture or piece of equipment within an animal enclosure, attached to fence(s) within a pasture, and so forth.

The veterinary acoustic and biofeedback device may include a housing in which components of the veterinary acoustic and biofeedback device are disposed. The housing may include any shape, such as an elongated cylindrical housing. The housing may be manufactured from any suitable materials, such as plastics, composites, metal, etc., and using any manufacturing process(es), such as injection molding, stamping, blow molding, etc. The housing may also be contaminate-resistant to prevent the ingress of liquid, dust, or contaminants into the housing. As introduced above, the housing may be configured to be worn by the animal, for example, through the use of a harness (e.g., collar), or may be separate from the animal.

As will be discussed herein, the veterinary acoustic and biofeedback device may include suitable processing and internal components to detect the animal welfare and behavior, as well as to treat, manage, and/or prevent pathological and/or physiological states. For example, the veterinary acoustic and biofeedback device may include one or more sensor(s), microphone(s), battery (ies), processing components (e.g., processors, memory, system on a chip (SoC) or integrated circuits (IC), etc.), etc. disposed within the housing. In an embodiment, the veterinary acoustic and biofeedback device may include I/O components, such as a display for outputting information associated with the animal. The display may include an organic light emitting diode (O/LED), (in-plane switching (IPS) or thin-film-transistor (TFT)) liquid crystal display (LCD), laser video display (LVD), vibrotactile, haptic, or other applications. In an embodiment, the device (or a component of a system) may even include a camera whereby still and or video image data may be collected for confirmation or collaboration in connection with the audio and/or other data gathered.

In an embodiment, a plurality of veterinary acoustic and biofeedback devices may be used in conjunction with one another, such as in a system, to determine the animal welfare and/or pathological, biological, and/or behavioral states. For example, a first veterinary acoustic and biofeedback device (i.e., a first system component) worn on the animal may be used in conjunction with a second veterinary acoustic and biofeedback device (i.e., a second system component) placed within a pasture. In such instances, the veterinary acoustic and biofeedback devices may be the same or different, for example, in size, functionality, capabilities, etc. For example, the first veterinary acoustic and biofeedback device may include first sensors that are different from the second sensors of the second veterinary acoustic and biofeedback device. In instances in which a plurality of the veterinary acoustic and biofeedback devices are used, the veterinary acoustic and biofeedback device(s) may be communicatively coupled to one another via one or more network interface(s) (e.g., Bluetooth, Cellular, etc.). Any combination of veterinary acoustic and biofeedback device(s) worn on the animal and/or off the animal may be used, or by different animals may be used.

The veterinary acoustic and biofeedback device may be used in association with any type of animal. For example, the animals may be any suitable animal, such as livestock (e.g., cattle, sheep, pigs, etc.), pets (e.g., horses, dogs, cats, parrots, etc.), wildlife subjects (e.g., wolves, whales, dolphins, waterfowl, birds, etc.), and so forth. In an embodiment, the veterinary acoustic and biofeedback device may be designed or preconfigured for the animal. For example, a size, type, material, etc. of a harness may be dependent upon the animal. In addition, the placement of the veterinary acoustic and biofeedback device on the animal may be dependent upon the type of animal. For example, to detect welfare, behavioral, and/or pathological states, the veterinary acoustic and biofeedback device may be placed at predetermined positions on the animal. For example, in bovine and ovine applications, the large animals tolerate a collar-type device, in which case acoustic sensing of laryngeal/vocal activity is conveniently proximal to the animal's larynx and airway physiology. Other applications, such as for poultry or waterfowl, may require leg-mounted devices with special attention to the weight of the overall device relative to animal size and proportion. In another application, penned or cloistered animals may be monitored with devices mounted to the penning structure. Free-range or grazing/roaming animals may require exclusively body-worn devices. These necessitated physical mounting demands may require adaptations to device characteristics such as power management, number of sensors, orientation of device, and so on.

The veterinary acoustic and biofeedback device may include any number of sensor(s) to monitor the animal and/or the environment of the animal for determining the welfare, behavioral, and/or pathological states. Example sensor(s) include but are not limited to, microphone(s), piezoelectric sensor(s), accelerometer(s), temperature sensor(s), heart rate sensor(s), humidity sensor(s), inertial measurement unit (IMU) sensor(s), gyroscope, GPS sensor(s), blood oxygen sensor(s), Hall sensor(s), passive infrared (PIR) sensor(s), etc. In an embodiment, the sensor(s) may be used to identify activity and/or non-activities of the animal, as well as to characterize the activity and/or non-activity. For example, the sensor(s) may be used to determine activities of the animal (e.g., vocalizations, respiration, movement, eating, sleeping, etc.) and/or non-activates of the animal (e.g., non-vocalizations, lameness, etc.). Moreover, the sensor(s) may be used to determine vocalizations and/or behavior of the animal for deducing the welfare, behavioral, and/or pathological states.

As will be discussed herein, the microphone(s) collect audio data, and the sensor(s) collect sensor data that is analyzed to determine the laryngeal/vocal biomarkers, physical biomarkers, behavioral biomarkers, environmental biomarkers, and/or other audio and physiological markers associated with the animal or an environment of the animal. The veterinary acoustic and biofeedback device may include analog and digital converters that assist in the capture, processing, and storage of signals sampled from the microphone(s) and sensor(s), respectively. The signals from the microphone(s) and sensor(s) may be stored in a dataset, including raw and/or normalized or transformed values. The audio signals or sensor signals may be processed as data, which is analyzed to determine the vocal biomarkers, physical biomarkers, behavioral data, environmental biomarkers, and/or other audio markers.

In an embodiment, the vocal biomarkers may be associated with vocalizations and laryngeal activity (e.g., sound, communications, respiratory activity, etc.) produced by the animals. In an embodiment, the vocalizations may be associated with pain (e.g., groaning, grunting, labored breathing, etc.), distress (e.g., predators in area), social (e.g., signaling isolation/separation of a calf from its mother, mating calls, nursing or mating sounds, etc.). In an embodiment, characteristics of the vocalizations may be determined for use in determining the welfare, behavioral, and/or pathological states. In doing so, the vocalizations may be used to determine a health of the animal for providing treatment and/or responding to the animal.

The vocalizations may be based at least in part on the type of animal and/or the environment of the veterinary acoustic and biofeedback device. In an embodiment, the vocal biomarkers may be determined based at least in part on characteristic(s) of the audio, such as tone, prosody, duration, amplitude, etc. In an embodiment, the audio data generated by the microphone(s) may be processed to determine the vocal biomarkers. Additionally, or alternatively, sensor data generated by the sensor(s) may be processed to determine the vocal biomarkers. For example, sound captured by the microphones and data indicative of inhaling, exhaling, or vocalizing (e.g., via vocal cord vibration, for example) generated by the sensor(s) may be processed to determine the vocal biomarkers.

In an embodiment, the physical biomarkers may be associated with physical activity of the animals, such as walking, calving, mating, avoidance behavior, inter-animal interactions, volitional activity including laryngeal vocalizations, searching or avoiding food or water sources, learned behaviors, etc. The environment biomarkers may be associated with weather prediction, seasonal variation, estimation of precipitation, identification of dangerous weather patterns (e.g., electrical storms, flooding, etc.), and existence in the environment of other hazards (e.g., proximity to traffic noise, predator sounds, rival animals fighting, etc.), humidity, presence of other animals, etc. The audio markers may be associated with physiological, anatomical, behavioral, or mental state of the animal. There is growing scientific literature and worldwide concern over the welfare of animals as indicated by their behavior, activity, and interactions. Both physical and mental health of animals are measured as a function of animal behavior both individually and within-group. Using predictors as mentioned herein, objective indicators are collected via sensors in the device, used in the classification/discrimination models, and interpreted as actionable information for the animal directly or for their caretakers. For example, increased lowing frequency and increased amplitude in a post-natal cow may indicate acute separation of cow and calf as perceived by the cow. This indication may be monitored by the device and constitute a cause for alerting a caregiver. Other biomarkers, such as individual or situational biomarkers, may be used to characterize the experiences, activities, behaviors, and actions of the animal. In an embodiment, the vocal biomarkers, physical biomarkers, environmental biomarkers, and/or other audio markers may be generally referred to herein as “animal biomarkers.”

As introduced above, the veterinary acoustic and biofeedback device may include the microphone(s) for collecting audio signals associated with vocalizations, sounds, etc., generated from the animal or sounds generated from other sources within the environment. In an embodiment, the microphone(s) may include at least two microphones spaced apart from one another within the housing. For example, the housing may include a first end and a second end spaced apart from the first end. A first of the microphones may be located closer to the first end than the second end, while a second of the microphones may be located closer to the second end than the first end. Any number of microphones, however, may be included within the veterinary acoustic and biofeedback device. In an embodiment, the microphone(s) may be used for determining a directionality (e.g., time of flight, velocimetry, time-delay analytics, etc.) of the signal and/or whether the signal corresponds to the sound generated from the animal wearing the veterinary acoustic and biofeedback device (e.g., as compared to background noise, other animals, predators, weather, etc.). For example, the microphone(s) may receive respective audio signals that are representative of sounds within the environment.

When worn by the animal, the microphones may be spaced apart by different distances from sound sources of the animal (e.g., the mouth of the animal, digestive system, etc.). For example, when the veterinary acoustic and biofeedback device represents a device that attaches as a collar, the microphones may be located at different distances from the mouth of the veterinary acoustic and biofeedback device to discern sound from the animal as compared to other sources of sound within the environment. In an embodiment, the veterinary acoustic and biofeedback device may employ suitable techniques to detect, segregate, label, and classify animal sounds or other vocalizations. For example, audio signatures may be used to correlate sounds of the animal and/or the environment with certain known sounds. Additionally, statistical likelihood pattern-matching models may also be employed using templates of known animal laryngeal vocal activity or by machine comparisons with known, idealized, or structured samples.

The audio signals generated by the microphones may be cross-correlated. For example, cross-correlation may apply between audio signals detected by the microphones to determine a time difference of arrival (TDOA) for the correlated signals. In other words, the differently spaced microphones from the mouth of the animal have different time of arrival (TOA) values. This allows the calculation of an angle from which the detected signals originate relative to a line intersecting the two microphones.

In operation, the sensor(s) of the veterinary acoustic and biofeedback device may collect signals or data that is analyzed to determine the animal biomarkers. For example, a microphone may collect audio signals, or audio data, that is analyzed to determine vocalizations, acoustics, sound, etc. of the animal. Vocalizations of the animal may include not only sounds generated via a mouth of the animal (e.g., moaning, grunting, etc.) but sound generated by digestion, body parts, foot-fall, etc. of the animal. Moreover, the microphones may capture sound or other acoustics of an environment of the animal (e.g., weather, sounds of other animals, etc.).

In an embodiment, the veterinary acoustic and biofeedback device may be configured to identify sounds emanating or generated from the animal, as compared to sounds generated from other sources within the environment of the animal. For example, the veterinary acoustic and biofeedback device may utilize the sensor(s) to determine the sounds of the animal, their amplitude, signal-to-noise ratio, and so forth. Sounds may also be associated with the animal, as compared to other sources of sound, based on detecting vocal vibrations by detecting changes in pressure, breathing, acceleration, temperature, strain, force, etc. Times associated with generating the audio data and the sensor data may be associated with one another to correlate the sensor data with the audio data.

In addition to acoustics, accelerometers may generate sensor signals or sensor data to determine behaviors of the animal, such as movements (e.g., bedding, running, calving, digestion, etc.). Other sensor(s) may be used to classify the behavior of the animal and the environment of the animal. For example, heart rate sensor(s) may be used to determine the health of the animal (e.g., electrocardiogram, photoelectric pulse wave, blood pressure measurement, phonocardiography, etc.), weather sensor(s) may be used to determine weather conditions (e.g., temperature of the animal and/or environment), and so forth. Based at least in part on the audio data and/or the sensor data, the animal biomarker(s) associated with the animal may be determined. Any combination of the audio data and/or the sensor data may be used to determine the animal biomarker(s) for determining the welfare, behavioral, and/or pathological states.

The animal biomarkers may be compared against reference animal biomarkers or thresholds, for example, to determine the animal welfare. In an embodiment, the pathological and/or behavioral states of the animal may be determined based at least in part on the animal welfare. In an embodiment, the reference biomarkers may be considered diagnostic/classificatory biomarkers in that the animal biomarkers are compared to the reference biomarkers to determine welfare, behavioral, and/or pathological details. As an example, poultry have been observed to express up to thirty distinct vocalizations corresponding to different pathological states. Capturing sounds of the animal allows for the determination of whether the poultry, for example, includes a pathological state that warrants care, treatment, etc. As such, sounds (e.g., pathological sounds) emitted from the animal may be detected and analyzed to identify and inform a caregiver regarding the pathological state of the animal, whether normal, suspect, or determined pathological or distress experienced by the animal. In an embodiment, these different vocalizations may have, or be characterized by, reference audio signatures. As will be discussed herein, audio signatures of the vocalizations of the animal may be compared against reference audio signatures to determine the welfare, behavioral, and/or pathological state of the animal.

Depending upon the type of animal, comparable sets of reference biomarkers may be used for determining the animal welfare, biological, behavioral, and/or pathological state. For example, the veterinary acoustic and biofeedback device may have reference biomarkers for cattle, dogs, birds, etc. for comparison to the animal biomarker(s). The comparison between the animal biomarkers and the reference animal biomarkers or thresholds may determine a statistical likelihood of pathological states of the animal. Moreover, the veterinary acoustic and biofeedback device may be preconfigured to monitor for certain types of welfare, behavioral, and/or pathological states. For example, the veterinary acoustic and biofeedback device may be preconfigured to monitor for fowl cholera within poultry, as compared to other diseases (e.g., salmonellosis). For example, bovine respiratory disease (BRD) or porcine respiratory disease complex (PRDC) may have specific laryngeal, vocal, and respiratory signatures and identifiable biomarkers identified by the device. In an embodiment, the veterinary acoustic and biofeedback device may be configured to monitor for any number of pathological states.

In an embodiment, the animal welfare may be associated with healthy, sick, diseased, etc. In an embodiment, animal welfare may be used to classify or determine the pathological states of the animal. Pathological states may be related to, but are not limited to, respiration, feeding, water intake, movement, digestive, gastrointestinal, mating or calving, disease, metabolism, gate (e.g., lameness, mastitis, metritis, ketosis, etc.), and so forth. These pathological states may be correspondingly utilized to identify, characterize, diagnose, and treat, etc. diseases (e.g., states, factors, etc.), disabilities, distresses, welfare disturbances, pathologies, activities, congenital defects, effects of medications, environmental effects, accidents, harmful physical events, etc. associated with the animal.

In an embodiment, the veterinary acoustic and biofeedback device may generate audio fingerprint(s) of the animals based at least in part on the animal biomarkers. The audio fingerprint(s) may be individual to the animals and permit the animals to be distinguished from one another, for example, to know which animal amongst a group of animals produced a sound or contributed data to the device's sensor array. This allows the sound to be associated with a certain animal to accurately determine the welfare, behavioral, and/or pathological state of a particular animal.

The vocal fingerprint or phenotype may also improve the accuracy of determining welfare, behavioral, and/or pathological states of the animal. For example, in an embodiment, the animal biomarkers may be used to generate audio fingerprint(s) associated with the animal, and the audio fingerprint(s) may be analyzed to determine whether the audio fingerprint(s) is associated with the particular behavioral and/or pathological state. In other words, the audio fingerprint(s) may characterize the sounds of the animal, and the audio fingerprint(s), for example, may be compared to one or more reference fingerprints to determine whether the animal is calving, running, ill with disease, having bowel discharge, etc., based on a comparison to reference fingerprints associated with the welfare, behavioral, and/or pathological states. In an embodiment, the use of the audio fingerprint(s) may discern sound generated from the animal from other sounds emanating within the environment.

In addition to audio fingerprint(s) of the animal, other audio and acoustic fingerprint/phenotype biomarkers may be used in the veterinary acoustic and biofeedback device. For example, characteristic environmental auditory events such as stampeding, weather (e.g., lighting), predators (e.g., howling), etc., have unique acoustic signatures that may be used in combination or solely to better interpret the auditory and acoustic environment of the veterinary acoustic and biofeedback device.

In an embodiment, the types of pathological states that are detected may be based at least in part on the characteristic(s) of the animal. For example, the animal biomarkers may be based at least in part on other characteristic(s) of the animal, such as age, sex, breed, species, etc. For cattle, diseases such as BVD, BVDV, and PRDC, among others, may be checked. In addition, a history of the animal may be accessed when determining or to determine the animal welfare, behavioral, and/or pathological states. For example, a disease state, audiological status, medical history, medications, etc. may be used to determine the behavior and/or pathological states of the animal.

In an embodiment, the veterinary acoustic and biofeedback device may output the notification(s) on devices, or transmit the notification(s) to other devices associated with the welfare, behavioral, and/or pathological states. In an embodiment, the notification(s) may be output in real-time or substantially real-time. In an embodiment, the notification(s) may be audible, visual, olfactory, electronic, haptic, and/or any combination thereof. For example, the veterinary acoustic and biofeedback device may include a speaker that outputs audio (e.g., deeps, tones, instructions, etc.), lighting elements that illuminate (e.g., patterns, colors, etc.), and/or a motor that vibrates, etc. By outputting the notification(s), the veterinary acoustic and biofeedback device may provide real-time alerts to the caregiver of the animal. For example, a caregiver tending to their animals may be notified as to the animal welfare, behavioral, and/or pathological states. In an embodiment, the notification(s) may be output when the welfare, behavioral, and/or pathological states are concerning, troublesome, etc. The caregiver, for example, may determine when, or how, the notification(s) are output. For example, the caregiver may specify that based on the welfare, behavioral, and/or pathological states, when the notifications are to be output. The veterinary acoustic and biofeedback device may output the notification(s) for a predetermined amount of time, continuously, and/or until the caregiver responds, aids, etc., the animal.

In addition to real-time feedback as described above, asynchronous reports may also be delivered to the caregiver and stored in the software. For example, a daily histography digest of performance and behavior may be delivered via email or software (e.g., a mobile application) to the caregiver for offline use. Performance data may also be delivered to a database collection of performance characteristics constituting the basis for additional software and algorithm changes. The application and/or software may be embedded into the hardware/device or may be external software capable of being instantiated into external hardware such as a mobile phone, tablet, or computer.

The veterinary acoustic and biofeedback device may continuously generate or receive signals from the microphone(s) and/or sensor(s) for determining the welfare, behavioral, and/or pathological states. This allows for the determination of continual and present behavioral and/or pathological states for use in determining whether the behavioral and/or pathological states are ongoing or have been resolved. In an embodiment, the veterinary acoustic and biofeedback device may determine the behavioral and/or pathological states in real-time or may determine the veterinary acoustic and biofeedback device over a period of time.

In an embodiment, the veterinary acoustic and biofeedback device may utilize artificial intelligence (AI) and machine learning (ML) models to improve the accuracy of determining the animal welfare, behavioral, and/or pathological states. To clarify, AI and ML techniques, which are described and detailed with examples below, are used as encompassing cover terms for “smart” and algorithmic collection, processing, analyzing, databasing, managing, summarizing, and using data in an automated and large-scale fashion. The ML model(s) may be trained from a database (e.g., historical data, such as audio data that includes respiration, feeding, water intake, movement, digestive, gastrointestinal, mating or calving, disease, metabolism, gait (e.g., lameness, mastitis, metritis, ketosis, etc.), disabilities, distresses, welfare disturbances, pathologies, activities, congenital defects, effects of medications, environmental effects, accidents, harmful physical events, etc. of the animal) to analyze the data captured by the sensor(s) and microphone(s) for identifying the animal biomarkers, determining the animal welfare, behavioral, and/or pathological states. The ML model(s) may determine the animal biomarkers and may assess the animal biomarkers in comparison to the information stored in the database (e.g., reference characteristics, reference vocal fingerprint, etc.) to determine the welfare, behavioral, and/or pathological states.

As part of the ML model(s) analyzing the audio data and/or the sensor data, the ML model(s) may label the characteristic(s) of the vocalizations and/or behavior of the animal, for example, the animal biomarkers, to indicate whether the characteristic(s) are associated certain animal welfare and/or pathological states. In an embodiment, an output of the ML model(s) may indicate whether the vocalizations and/or behavior are associated with a particular animal welfare and/or pathological state (e.g., healthy, diseased, calving, injured, etc.). In an embodiment, the ML model(s) may determine the characteristic(s) for comparison to respective references for determining whether the vocalizations and/or behavior are indicative of, or are not indicative of, a particular welfare, behavioral, and/or pathological state. The ML model(s) may use any number of characteristic(s) for determining whether the vocalizations and/or behavior are indicative of, or are not indicative of, the particular welfare, behavioral, and/or pathological state.

In an embodiment, the ML model(s) may generate scores that are associated with the characteristic(s) of the welfare, behavioral, and/or pathological states. For example, the ML model(s) may generate scores associated with the animal biomarkers, animal welfare, behavioral, and/or pathological states. If the scores are greater than a predetermined threshold (which varies depending on the particular data and desired information relayed in return), the ML model(s) may have a threshold confidence that the characteristics are associated with a particular pathological state of the animal, for example. In an embodiment, the ML model(s) may compare an audio fingerprint(s) of the animal with a reference audio fingerprint. A pathological state may be assessed with regard to the model parameters, and a discrete or probabilistic classification will be generated. The classification may be stored, processed, and used for feedback (as needed).

Although the veterinary acoustic and biofeedback device is described as processing, analyzing, and/or determining the animal biomarkers, welfare, behavioral, and/or pathological states, etc., in an embodiment, the veterinary acoustic and biofeedback device may communicatively couple to one or more computing devices, resource(s), etc. which may additionally or alternatively process, analyze, and/or determine the animal biomarkers, animal welfare, pathological states, etc. For example, the veterinary acoustic and biofeedback device may receive sensor data that is transmitted to remote computing resource(s) for analysis. As introduced above, the remote computing resource(s) may receive the sensor data from any number of the veterinary acoustic and biofeedback device(s), separate device(s) (e.g., weather and/or temperature device), etc.). In an embodiment, the remote computing resource(s) may analyze the sensor data, among other data, to determine the animal biomarkers, welfare, behavioral, and/or pathological states, etc. Thereafter, the remote computing resource(s) may transmit notification(s) to the veterinary acoustic and biofeedback device, the device of the caregiver, and so forth. Any level of split processing may be performed by the veterinary acoustic and biofeedback device, the device of the caregiver, the remote computing resource(s), and/or other devices, systems, networks, etc.

The present disclosure provides an overall understanding of the principles of the structure, function, device, and system disclosed herein. One or more examples of the present disclosure are illustrated in the accompanying drawings. Those of ordinary skill in the art will understand and appreciate that the devices, the systems, and/or the methods specifically described herein and illustrated in the accompanying drawings are non-limiting embodiments. The features illustrated or described in connection with one embodiment, or instance, may be combined with the features of other embodiments or instances. Such modifications and variations are intended to be included within the scope of the disclosure and appended claims.

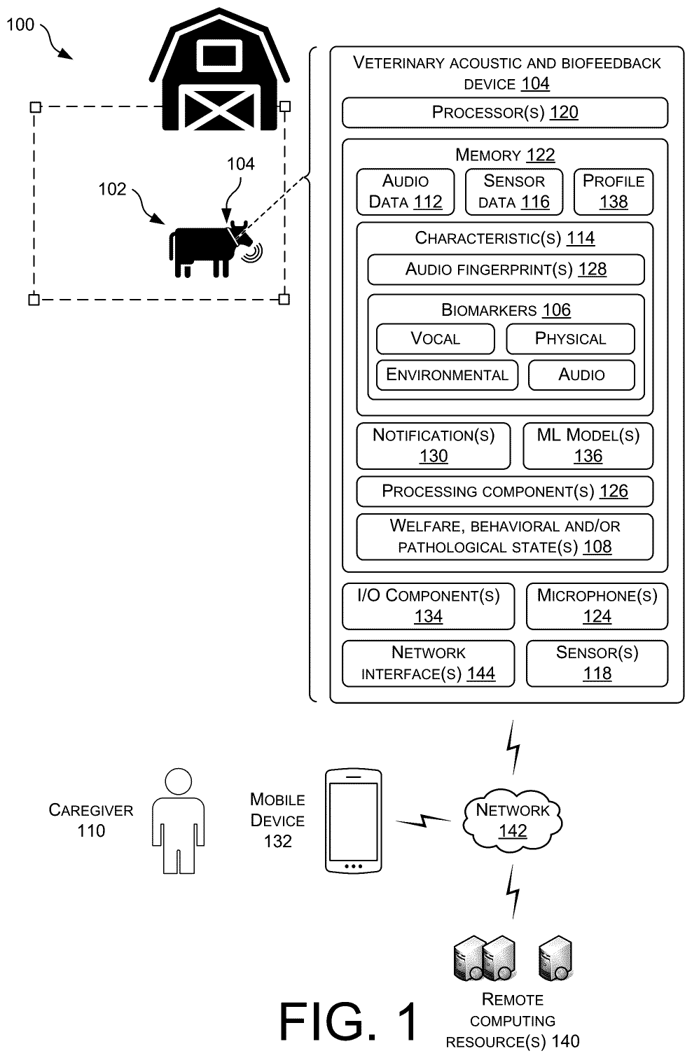

FIG. 1 illustrates an example environment 100 including an animal 102 wearing a veterinary acoustic and biofeedback device 104, according to examples of the present disclosure. Details of the veterinary acoustic and biofeedback device 104 are discussed herein, however, generally, the veterinary acoustic and biofeedback device 104 may determine biomarkers 106 of the animal 102 for detecting a welfare, behavioral, and/or pathological states 108 of the animal 102. Detection of the welfare, behavioral, and/or pathological states 108 allows for a caregiver 110 (e.g., owner, veterinarian, rancher, etc.) to treat and/or manage the animal 102.

The veterinary acoustic and biofeedback device 104 may be a compact, wearable, or peripheral device. In an embodiment, the veterinary acoustic and biofeedback device 104 may be worn on a body of the animal 102. In an embodiment, the veterinary acoustic and biofeedback device may be small, non-invasive, non-intrusive, lightweight, and discreet in nature. As shown in FIG. 1, the veterinary acoustic and biofeedback device 104 may be worn as a collar around a neck of the animal 102. However, in an embodiment, the veterinary acoustic and biofeedback device 104 may be embodied within a harness, stick-on arrays, saddle, tag, etc. that is worn by the animal 102. Additionally, or alternatively, the veterinary acoustic and biofeedback device 104 may be attached or worn by the animal 102 using attachment mechanisms, such as hook and loop, clasps, magnets, cords, chains, clips, belts and buckles, cords, pins, adhesives, etc. for coupling (e.g., pinning, hanging, etc.) in any manner to the animal 102. Still, in an embodiment, the veterinary acoustic and biofeedback device 104 may be sub-dermally implanted, injected, or ingested.

In an embodiment, the veterinary acoustic and biofeedback device 104, (and/or, as discussed above, a component of a system), may be separate from the animal 102 (i.e., not worn by the animal 102), placed in proximity to the animal 102. For example, the animal 102 may be housed in a barn, stable, bed, pen, cage, etc. Here, the veterinary acoustic and biofeedback device 104 may be positioned relative to the animal 102, or a group of animals, to monitor the animal 102 or the environment 100 of the animal 102. In an embodiment, the veterinary acoustic and biofeedback device 104 may be mounted on a wall or door of a pen, for example, attached to a fixture or piece of equipment within an animal enclosure, attached to fence(s) within a pasture, and so forth.

The veterinary acoustic and biofeedback device 104 may be used in association with any type of animal 102. For example, as shown in FIG. 1, the veterinary acoustic and biofeedback device 104 may be used in conjunction with a cow. However, other suitable animals may include livestock (e.g., sheep, pigs, etc.), pets (e.g., horses, dogs, cats, parrots, etc.), wildlife subjects (e.g., wolves, whales, dolphins, waterfowl, birds, etc.), and so forth. In an embodiment, the veterinary acoustic and biofeedback device 104 may designed or preconfigured for the animal 102. For example, a size, type, material, etc. of a harness may be dependent upon the animal 102. Moreover, in an embodiment, the animal 102 may be free to roam, graze, traverse within the environment 100, or may be housed within a barn, within fences, etc. In either scenario, the veterinary acoustic and biofeedback device 104 may be configured to monitor the animal 102 for the welfare, behavioral, and/or pathological states 108, and may utilize communication functions compatible with remote transmission componentry dispersed in the region where the animal 102 inhabits.

The veterinary acoustic and biofeedback device 104 may capture, generate, or receive audio data 112, such as vocalizations, sound, noises, etc. of the animal 102 or the environment 100 of the animal 102 and process the audio data 112 to detect the welfare, behavioral, and/or pathological states 108. Based on processing the audio data 112, the veterinary acoustic and biofeedback device 104 may provide real-time feedback to the caregiver 110 that prompts the caregiver 110 to treat or manage the animal 102, for example, based at least in part on the welfare, behavioral, and/or pathological states 108. For example, the caregiver 110 may provide assistant to the animal 102, administer vaccines, medications, etc. In an embodiment, the veterinary acoustic and biofeedback device 104 may continuously capture and process the audio data 112 to provide real-time feedback and further may incorporate additional video data where video may be real-time, constantly streaming, or triggered to record by a signal from the veterinary acoustic and biofeedback device 104.

In an embodiment, the veterinary acoustic and biofeedback device 104 may process the audio data 112 to determine characteristic(s) 114 of the vocalizations of the animal 102. The characteristic(s) 114 may be compared to reference characteristics, thresholds, etc. for detecting when the vocalizations are indicative of certain welfare, behavioral, and/or pathological states 108 or include characteristic(s) associated with certain welfare, behavioral, and/or pathological states 108. In an embodiment, the characteristic(s) 114 may include or be represented by biomarkers 106, where the biomarkers 106 may identify or be associated with the characteristic(s) 114. As an example, the veterinary acoustic and biofeedback device 104 may detect one or more of the biomarkers associated with BVD or with PRDC or with Influenza A Virus (IAV) including subtypes such as H5N1, H5N3, and H7N9 for determining the welfare, behavioral, and/or pathological states 108. For example, in domesticated poultry or wild avian applications, respiratory pathology or irregularities could be detected by the veterinary acoustic and biofeedback device and software and reported to caretakers to promote action including vaccination or culling of infected individuals. In another example, the laryngeal and vocal productions of swine infected with PRDC could be monitored for symptomatic labored breathing, irregular ingressive and egressive respiration rates, foreign substance (such as mucous) expulsion, and so forth to determine the welfare, pathology, and behavior of animals. Increased probability of symptomatic laryngeal activity could be coupled with geographical location or inter-individual location tracking to estimate the likelihood of disease transmission and afford the caretakers or others opportunity to intervene or track changes in behavior, health, or well-being.

In an embodiment, the biomarkers 106 may include vocal biomarkers, physical biomarkers, environmental biomarkers, and/or audio biomarkers. In an embodiment, the vocal biomarkers may be associated with vocalizations (e.g., sound, communications, etc.) produced by the animal 102. In an embodiment, the vocalizations may be associated with any sounds generating or emanating from the animal 102, such as pain (e.g., groaning), distress (e.g., predators in area), social (e.g., signaling isolation/separation of a calf from its mother, mating calls, nursing or mating sounds, etc.), flatulence and excretion of solid and liquid animal waste, and so forth. In an embodiment, the vocal biomarkers may be determined based at least in part on characteristic(s) of the audio, such as tone, prosody, duration, amplitude, etc. In an embodiment, the physical biomarkers may be associated with physical activity of the animals, such as walking, calving, mating, voiding of waste, etc. The environment biomarkers may be associated with weather prediction, estimation of precipitation, identification of dangerous weather patterns (e.g., electrical storms, flooding, etc.), and existence in the environment of other hazards (e.g., proximity to traffic noise, predator sounds, rival animals fighting, etc.), humidity, etc. The audio markers may be associated with physiological, anatomical, behavioral, or mental state of the animal 102. The welfare of animals is indicated by their behavior, activity, and interactions. The physical and mental health of animals are measured as a function of animal behavior both inter- and intra-individually. Using predictors as mentioned herein, objective indicators are collected via the sensor(s) 118 of the veterinary acoustic and biofeedback device 104, used in the classification/discrimination models, and interpreted as actionable information for the animal 102 directly or for the caregiver 110. For example, increased lowing frequency and increased amplitude in a post-natal cow may indicate acute separation of cow and calf as perceived by the cow. This indication may be monitored by the veterinary acoustic and biofeedback device 104 and constitute cause for alerting the caregiver 110. Other biomarkers, such as individual or situational biomarkers, may be used to characterize experiences, activities, behaviors, and actions of the animal 102.

In an embodiment, the biomarkers 106 may be determined based at least in part on the audio data 112 generated by microphone(s) 124. The audio data 112 includes but it not limited to spectral and temporal analyses of the sampled signal including fundamental frequency, harmonics of vocal tract including formant frequencies, respiratory support, amplitude characteristics, source-filter characteristics of vocal fold and laryngeal activity coupled to upper airway morphophysiology and anatomy, and vocal amplitude. These features are characterized and interpreted in combination, to infer physiological details of the animal 102 (e.g., vocal tract length, oral aperture, laryngeal port motor control, lingual shape characteristics, etc.), physio-temporal details (e.g., fine- and gross-motor control, etc.), and range/tendency/error/entropy/stability in the observed distributions in the signal. Additionally, or alternatively, sensor data 116 generated by sensor(s) 118 may be processed to determine the biomarkers 106. For example, sound captured by the microphone(s) 124 and data indicative of inhaling, exhaling, and vocalizing (e.g., via vocal cord vibration, for example) generated by the sensor(s) 118 may be processed to determine the biomarkers 106.

The veterinary acoustic and biofeedback device 104 is shown including processor(s) 120 and memory 122, where the processor(s) 120 may perform various functions and operations associated with detecting the welfare, behavioral, and/or pathological states 108, and the memory 122 may store instructions executable by the processor(s) 120 to perform the operations described herein. The veterinary acoustic and biofeedback device 104 may include any number of the sensor(s) 118 for detecting the vocalizations, behavior, etc. of the animal to determine the welfare, behavioral, and/or pathological states 108. For example, using the sensor(s) 118, the veterinary acoustic and biofeedback device 104 may determine the biomarkers 106 associated with the animal 102.

In an embodiment, the microphone(s) 124 may include at least two microphones. Any number of microphone(s) 124, however, may be included within the veterinary acoustic and biofeedback device 104. The microphone(s) 124 may be used for determining a directionality (e.g., time of flight, time-delay analytics, etc.) of sound and/or whether the sound corresponds to the animal 102 and/or other sources (e.g., other animals, weather, etc.). The microphone(s) 124 may generate respective audio signals that are representative of sound within the environment 100. In an embodiment, the microphone(s) 124 may be spaced apart by a different distance from a mouth of the animal 102, for example, to discern sounds that are generated by the animal 102 from other sources of sound within the environment 100.

The veterinary acoustic and biofeedback device 104 may include processing component(s) 126, such as echo cancellation, noise reduction, beamforming, and the like, to enable processing of the vocalizations and behavior of the animal 102. In an embodiment, the processing component(s) 126 may include a beamforming module used to process audio signals generated by the microphone(s) 124. Beamforming or spatial filtering is a signal processing technique for directional signal reception. Signals generated by the microphone(s) 124 may be processed in such a way that signals at particular angles experience constructive interference while others experience destructive interference. The beamforming techniques form multiple directional signals corresponding to different directions or orientations within the environment 100.

In an embodiment, the veterinary acoustic and biofeedback device 104 may self-adjust or calibrate (in real-time or over a course of repeated use sessions). Calibration may yield more sensitive or situationally adjusted results when determining the welfare, behavioral, and/or pathological states 108. The calibration and sensitization may operate by combining, for example, detection and coordinate processing of amplitude for an animal to refine or otherwise modify subsequent detection of the welfare, behavioral, and/or pathological states 108.

In an embodiment, other examples of the sensor(s) 118 of the veterinary acoustic and biofeedback device 104, such as piezoelectric sensors, accelerometers, etc., may generate signals that are processed to generate sensor data 116 that are used to determine the biomarkers 106. The sensor(s) 118 may additionally or alternatively include global positioning satellite (GPS), gyroscope(s), inertial measurement units (IMU), etc. The sensor data 116 generated from the sensor(s) 118, such as an IMU, may be used to determine vocalizations of the animal, for example, based on experienced vibrations, accelerations, inhaling/exhaling, pressure changes, and so forth. The sensor(s) 118 may be calibrated to optimize function and increase processing fidelity. The sensor data 116 collected by the sensor(s) 118 may be used in combination with the audio data 112. Timestamps associated with the audio data 112 and the sensor data 116 may be associated with one another to correlate the sensor data 116 with the audio data 112. In an embodiment, to effectively detect vocalization, the sensor(s) 118 may be positioned in proximity to the clavicle (or furcula in birds, etc.) or thyroid/cricoid area (or equivalent), or near vocal production mechanism of the animal 102 to better detect movement or pressure changes associated with vocalization and/or respiration.

The audio data 112 and/or sensor data 116 may be analyzed to determine the biomarkers 106. In an embodiment, the biomarkers 106 may be associated with the vocalizations of the animal 102, such as pitch, intonation, tone, pauses, phonation, energy changes in the harmonic spectrum, fundamental frequency, voicing, windowed and absolute sonorant-peak rates, SNR, temporal and spectral voice characteristics, frequency, spectral/cepstral cues, vocal quality/stability, prosody, temporal output, and amplitude stability, and/or motor processes. In an embodiment, the biomarkers 106 may be characterized by duration, frequency, decibel level, amplitude, energy, etc.

The biomarkers 106 may also be associated with movements of the animal 102 (e.g., bedding, running, walking, etc.), activities of the animal 102 (e.g., calving, drinking, eating, etc.), the environment (e.g., weather, temperature, etc.), etc.

In an embodiment, the veterinary acoustic and biofeedback device 104 may process the audio data 112 and/or the sensor data 116 to determine the biomarkers 106 for use in detecting the welfare, behavioral, and/or pathological states 108, such as diseases, distress, etc. The vocalizations of the animal 102 may be used to deduce the welfare, behavioral, and/or pathological states 108, or stated alternatively, the vocalizations of the animal 102 may be compared against characteristics indicative of the welfare, behavioral, and/or pathological states 108. In addition, the activities or non-activities of the animal 102 may be used to determine the welfare, behavioral, and/or pathological states 108, or whether the behavior of the animal 102 is indicative of the welfare, behavioral, and/or pathological states 108. In an embodiment, the biomarkers 106 may be compared to one or more predetermined thresholds or reference characteristic(s) indicative of the welfare, behavioral, and/or pathological states 108, for example, to determine whether the behavior of the animal 102 is indicative of a certain pathological state. In this manner, the biomarkers 106 may be compared to reference characteristics of animals with the welfare, behavioral, and/or pathological states 108 and without the welfare, behavioral, and/or pathological states 108. Through this comparison, a statistical likelihood may be determined whether the vocalizations and/or behavior of the animal 102 is indicative of the welfare, behavioral, and/or pathological states 108.

In an embodiment, the veterinary acoustic and biofeedback device 104 may determine audio fingerprint(s) 128 (or phenotype). The use of the audio fingerprint(s) 128 may increase an accuracy in detecting vocalizations of the animal 102, discerning a type of vocalizations of the animal 102, or discerning other sounds emanating within the environment 100. Moreover, the audio fingerprint(s) 128 may be used to more accurately detect the biomarkers 106, and/or the welfare, behavioral, and/or pathological states 108. For example, in an embodiment, the biomarkers 106 may be used to generate audio fingerprint(s) 128 (e.g., audio signatures, acoustic fingerprint, etc.) associated with the animal 102, and the audio fingerprint(s) 128 may be analyzed to determine whether the audio fingerprint(s) 128 is associated with, or includes, characteristics of the welfare, behavioral, and/or pathological states 108. In an embodiment, the audio fingerprint(s) 128 may be compared to one or more reference fingerprints to detect the welfare, behavioral, and/or pathological states 108. Capturing sounds of the animal 102 allows for the determination of whether the poultry, for example, includes a pathological state that warrants care, treatment, etc. Each of the audio fingerprint(s) 128 may indicate biomarkers 106, and if a similarity between the audio fingerprint(s) 128 and a stored reference audio voiceprint exists, the welfare, behavioral, and/or pathological states 108 may be detected. The audio fingerprint(s) 128 may be compared to any reference audio voiceprint for determining whether any number of welfare, behavioral, and/or pathological states 108 exist.

In an embodiment, the veterinary acoustic and biofeedback device 104 may output notification(s) 130 in instances where welfare, behavioral, and/or pathological states 108 are detected. The notification(s) 130 may provide feedback to the caregiver 110 such that the caregiver 110 may administer aid, assist, treat, etc. the welfare, behavioral, and/or pathological state 108. For example, if the animal 102 has a disease, the caregiver 110 may administer medicine or other intervention. The notification(s) 130 may be output in real-time, or substantially real-time, to provide near-instantaneous feedback to the caregiver 110. In an embodiment, the notification(s) 130 may be associated with the biomarkers 106 that are detected. This may permit the caregiver 110 to make informed decisions about the administered care, or whether to not to administer care. In an embodiment, the notification(s) 130 may be output on a mobile device 132 of the caregiver 110 and/or at the veterinary acoustic and biofeedback device 104.

In an embodiment, the notification(s) 130 may be audible, visual, haptic, and/or a combination thereof. The veterinary acoustic and biofeedback device 104 may include I/O component(s) 134 that output the notification(s) 130. For example, lighting elements (e.g., LEDs) may output light at different brightness levels, flashing at different durations, frequencies and/or specific sequences (e.g., numeric sequences of long and short flashes) in different modes, and/or by lighting in different colors. Speaker(s) may output an audible signal, such as a continuous prompt tone, sounding at different pitches or loudness in different modes, sounding at different durations, frequencies and/or specific sequences (e.g., numeric sequences of loud and soft, or high and low pitch sounds) in different modes. In an embodiment, these notification(s) 130 may assist in locating the animal 102 within the environment 100, and/or deducing which animal 102 amongst a group (e.g., herd, flock, etc.) of animals needs treatment. Other output components of the veterinary acoustic and biofeedback device 104 may include displays, touchscreens, etc. Example input devices may include button(s), switches, toggles, etc.

In an embodiment, the veterinary acoustic and biofeedback device 104 may store or have access to machine-learned (ML) model(s) 136. The ML model(s) 136 may be trained to analyze the audio data 112 and/or the sensor data 116 to determine whether the welfare, behavioral, and/or pathological states 108 of the animal 102.

In an embodiment, the ML model(s) 136 may determine the biomarkers 106 for determining the welfare, behavioral, and/or pathological states 108 of the animal 102. In an embodiment, the ML model(s) 136 may determine or generate score(s) associated with the audio data 112 and/or the sensor data 116, to determine whether the audio data 112 and/or the sensor data 116 are indicative of certain welfare, behavioral, and/or pathological states 108. In an embodiment, the score(s) may relate to a probability or likelihood that the vocalizations and/or behavior of the animal 102 is associated with certain welfare, behavioral, and/or pathological states 108. In other words, the score(s) output by the ML model(s) 136 may be machine-learned scores. The welfare, behavioral, and/or pathological states 108 may be based at least in part on the type of animal 102.

Machine learning generally involves processing a set of examples (called “training data”) to train a machine learning model(s). A machine learning model(s), once trained, is a learned mechanism that can receive new data as input and estimate or predict a result as output. For example, a trained machine learning model may comprise a classifier that is tasked with classifying unknown input (e.g., an unknown audio) as one of multiple class labels. In some cases, a trained machine learning model is configured to implement a multi-label classification task. Additionally, or alternatively, a trained machine learning model may be trained to infer a probability or a set of probabilities, for a classification task based on unknown data received as input. In the context of the present disclosure, the unknown input may be the audio data 112 and/or the sensor data 116 that is associated with the animal 102, and the ML model(s) 136 may be tasked with outputting the score that indicates or otherwise relates to, a probability of the animal 102 having a particular welfare, behavioral, and/or pathological state 108. Additionally, the scores may indicate or otherwise relate to a probability of the biomarkers 106 for determining whether the vocalizations and/or behavior of the animal 102 is associated with the welfare, behavioral, and/or pathological states 108. Other data, such as medications of the animal 102, a history of the animal 102, etc. may be provided as an input to the ML model(s) 136.

The training data that is used to train ML model(s) 136 may include various types of data. In general, training data for machine learning may include two components, features and labels. However, in an embodiment, the training data used to train the ML model(s) 136 may be unlabeled. Accordingly, the ML model(s) 136 may be trainable using any suitable learning technique, such as supervised learning, unsupervised learning, semi-supervised learning, reinforcement learning, and so on. The features included in the training data may be represented by a set of features, such as in the form of an n-dimensional feature vector of quantifiable information about an attribute of the training data. The following is a list of example features that can be included in the training data for training the ML model(s) 136 described herein. However, it is to be appreciated that the following list of features is non-exhaustive, and features used in training may include additional features not described herein, and, in some cases, some, but not all, of the features listed herein. Example features included in the training data may include, without limitation, pitch, intonation, tone, pauses, articulation, decreased energy in the harmonic spectrum, fundamental frequency, SNR, temporal and spectral vocal characteristics, frequency, spectral/cepstral cues, vocal quality/stability, prosody, temporal output, and amplitude stability, and/or motor processes. In an embodiment, the features included within the training data may be associated with vocalizations and/or behavior of the animal 102 that contains welfare, behavioral, and/or pathological states 108 and/or does not contain welfare, behavioral, and/or pathological states 108.

In an embodiment, as part of the training process, weights may be applied to a set of features included in the training data, as derived from the historical data. In an embodiment, the weights that are set during the training process may apply to parameters that are internal to the ML model(s) 136 (e.g., weights for neurons in a hidden layer of a neural network). These internal parameters of the ML model(s) 136 may or may not map one-to-one with individual input features of the set of features. The weights may indicate the influence that any given feature, parameter, or characteristic has on the score that is output by the ML model(s) 136.

The ML model(s) 136 represent a single model or an ensemble of base-level machine learning models and may be implemented as any type of machine learning model. For example, suitable machine learning models for use with the techniques and systems described herein include, without limitation, neural networks, tree-based models, support vector machines (SVMs), kernel methods, random forests, splines (e.g., multivariate adaptive regression splines), hidden Markov model (HMMs), Kalman filters (or enhanced Kalman filters), Bayesian networks (or Bayesian belief networks), expectation maximization, genetic algorithms, linear regression algorithms, complex networks, nonlinear regression algorithms, logistic regression-based classification models, or an ensemble thereof. An “ensemble” can comprise a collection of machine learning models whose outputs (predictions) are combined, such as by using weighted averaging or voting. The individual machine learning models of an ensemble can differ in their expertise, and the ensemble can operate as a committee of individual machine learning models that is collectively “smarter” than any individual machine learning model of the ensemble.

In an embodiment, and as noted above, the ML model(s) 136 may identify or determine the characteristic(s) 114, such as the biomarkers 106, for comparison against the reference characteristic(s). The ML model(s) 136 may learn to identify complex relationships between the biomarkers 106 for determining whether the vocalizations and/or behavior is associated with the welfare, behavioral, and/or pathological states 108. For example, the ML model(s) 136 may learn to associate certain biomarkers 106 with whether the animal 102 has, or does not have, a certain welfare, behavioral, and/or pathological state 108.

In an embodiment, the ML model(s) 136 may learn to predict that the vocalizations and/or behavior is indicative of a welfare, behavioral, and/or pathological state 108 by attributing corresponding score(s). In this manner, biomarkers 106, for example, with low scores (e.g., below threshold) may not include a particular welfare, behavioral, and/or pathological states 108, while the biomarkers 106 with high scores (e.g., above threshold) may include a particular welfare, behavioral, and/or pathological states 108. Although the use of a threshold is described as one example way of providing labeling (i.e., replaced or maintained), other techniques are contemplated, such as clustering algorithms, or other statistical approaches that use the trust scores may be used. The ML model(s) 136 is/are retrainable with new data to adapt the ML model(s) 136.

In addition to processing the audio data 112 and/or the sensor data 116 to detect the welfare, behavioral, and/or pathological states 108, the veterinary acoustic and biofeedback device 104 may utilize other data. For example, the veterinary acoustic and biofeedback device 104 may have access to data stored within a profile 138. The data may indicate demographics of the animal 102, environmental factors, medications of the animal 102, etc. This data may additionally, or alternatively be used to treat, detect, manage, and/or prevent the welfare, behavioral, and/or pathological states 108. For example, incorporating user-specific, treatment-related, non-vocal data may be used to more effectively treat and manage the welfare, behavioral, and/or pathological states 108.

The biomarkers 106 may be stored in association with the profile 138. The profile 138 may also store the audio data 112 and/or the sensor data 116 in association with the animal 102. Such data may be used by the caregiver 110 to further refine therapeutic benefits made possible by the veterinary acoustic and biofeedback device 104. The audio data 112 and/or sensor data 116 may also be stored in the memory 122 of the veterinary acoustic and biofeedback device 104 and provided (e.g., downloaded) to other devices.

As shown, the veterinary acoustic and biofeedback device 104 may be communicatively coupled to one or more devices, such as the mobile device 132 and/or remote computing resource(s) 140 over network(s) 142. The network(s) 142 may include any viable communication technology, such as wired and/or wireless modalities and/or technologies. The network(s) 142 may include any combination of Personal Area Networks (PANs), Local Area Networks (LANs), Campus Area Networks (CANs), Metropolitan Area Networks (MANs), extranets, intranets, the Internet, short-range wireless communication networks (e.g., ZigBee, Bluetooth, etc.) Wide Area Networks (WANs)—both centralized and/or distributed—and/or any combination, permutation, and/or aggregation thereof. The veterinary acoustic and biofeedback device 104 includes suitable network interface(s) 144 for communicating over the network(s) 142.

In an embodiment, the veterinary acoustic and biofeedback device 104 may send the audio data 112 and/or the sensor data 116 to the remote computing resource(s) 140 for processing. The remote computing resource(s) 140 may receive the audio data 112 and/or the sensor data 116 from any number of veterinary acoustic and biofeedback device(s) 104 disposed within the environment 100, or other environments. The remote computing resource(s) 140 may have access to the components (e.g., ML model(s) 136) or additional components for processing the audio data 112 and/or the sensor data 116. In an embodiment, the remote computing resource(s) 140 may process the audio data 112 and/or the sensor data 116, determine the biomarkers 106, determine the welfare, behavioral, and/or pathological states 108, and then communicate the notification(s) 130 to the veterinary acoustic and biofeedback device 104, the mobile device 132, or other devices. Any level of split processing may be performed by the veterinary acoustic and biofeedback device 104, the mobile device 132, the remote computing resource(s) 140, and/or other devices, systems, networks, etc.

The remote computing resource(s) 140 may be implemented as one or more servers and may, in an embodiment, form a portion of a network-accessible computing platform implemented as a computing infrastructure of processors, storage, software, data access, etc. that is maintained and accessible via a network such as the Internet. The remote computing resource(s) 140 does not require end-user knowledge of the physical location and configuration of the system that delivers the services. Common expressions associated with the remote computing resource(s) 140 may include “on-demand computing”, “software as a service (SaaS)”, “platform computing”, “network-accessible platform”, “cloud services”, “data centers”, etc.

As used herein, a processor, such as the processor(s) 120 may include multiple processors and/or a processor having multiple cores. Further, the processor(s) 120 may comprise one or more cores of different types. For example, the processor(s) 120 may include application processor units, graphic processing units, and so forth. In one implementation, the processor(s) 120 may comprise a microcontroller and/or a microprocessor. The processor(s) 120 may include a graphics processing unit (GPU), a microprocessor, a digital signal processor or other processing units or components known in the art. Alternatively, or in addition, the functionally described herein can be performed, at least in part, by one or more hardware logic components. For example, and without limitation, illustrative types of hardware logic components that may be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) 120 may possess its own local memory, which also may store program components, program data, and/or one or more operating systems.

Memory, such as the memory 122 may include volatile and nonvolatile memory, removable and non-removable media implemented in any method or technology for storage of information, such as computer-readable instructions, data structures, program component, or other data. Such memory may include but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory may be implemented as computer-readable storage media (“CRSM”), which may be any available physical media accessible by the processor(s) to execute instructions stored on the memory. In one basic implementation, CRSM may include random access memory (“RAM”) and Flash memory. In other implementations, CRSM may include but is not limited to, read-only memory (“ROM”), electrically erasable programmable read-only memory (“EEPROM”), or any other tangible medium that can be used to store the desired information and which can be accessed by the processor(s) 120. The memory 122 is an example of non-transitory computer-readable media. The memory 122 may store an operating system and one or more software applications, instructions, programs, and/or data to implement the methods described herein and the functions attributed to the various systems.

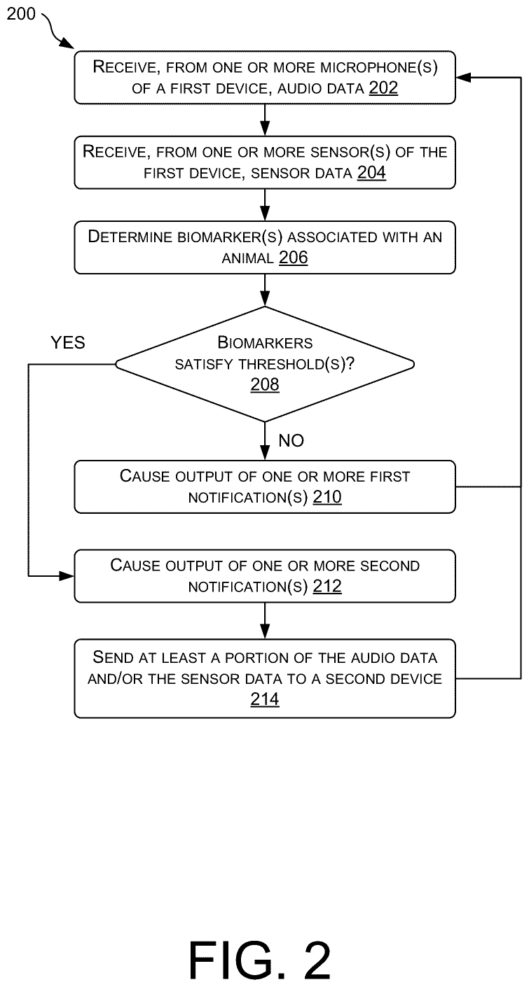

FIG. 2 illustrates an example process 200 (e.g., method) related to determining whether vocalizations and/or behavior of the animal 102 are associated with certain welfares, behavioral, and/or pathological states 108, according to examples of the present disclosure. The process 200 described herein is illustrated as collections of blocks in logical flow diagrams, which represent a sequence of operations or acts, some or all of which may be implemented in hardware, software, or a combination thereof. In the context of software, the blocks may represent computer-executable instructions stored on one or more computer-readable media that, when executed by one or more processors, program the processors to perform the recited operations or acts. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process 200 or alternative processes, and not all of the blocks need be executed. For discussion purposes, the process 200 is described with reference to the environments, devices, architectures, diagrams, and systems described in the examples herein, such as, for example those described with respect to FIG. 1, although the process 200 may be implemented in a wide variety of other environments, architectures, and systems.

At 202, the process 200 may include receiving audio data from one or more microphone(s) of a first device. For example, the veterinary acoustic and biofeedback device 104 may receive the audio data 112 as generated from the microphone(s) 124. In an embodiment, the audio data 112 may be at least partially processed or unprocessed (e.g., filtered). In an embodiment, rather than receiving audio data 112, the veterinary acoustic and biofeedback device 104 may receive audio signals.

At 204, the process 200 may include receiving sensor data from one or more sensor(s) of the first device. For example, the veterinary acoustic and biofeedback device 104 may receive the sensor data 116 as generated from the sensor(s) 118. In an embodiment, the sensor data 116 may be at least partially processed or unprocessed (e.g., filtered). In an embodiment, rather than receiving sensor data 116, the veterinary acoustic and biofeedback device 104 may receive sensor signals from the sensor(s) 118. The sensor(s) may include accelerometer(s), piezoelectric sensor(s), IMUs, GPS, etc. The veterinary acoustic and biofeedback device 104 may receive the sensor data 116 from any number of sensor(s) 118, and/or the number of sensor(s) 118 that generate the sensor data 116 may be based at least in part on an application of the veterinary acoustic and biofeedback device 104, the animal 102, a welfare, behavioral, and/or pathological state 108 being examined, etc.

At 206, the process 200 may include determining biomarker(s) associated with the animal. In an embodiment, the biomarker(s) 106 may be associated with determining whether welfare, behavioral, and/or pathological states 108 are detected. Any number of the biomarkers 106 may be determined, and the biomarkers 106 may be associated with vocalizations of the animal 102, activity of the animal 102, an environment of the animal 102, etc. As non-limiting examples, the biomarkers 106 may include a pitch of vocalizations of the animal 102, intonations of vocalizations of the animal 102, tones associated with the vocalizations of the animal 102, changes associated with the vocalizations of the animal 102, weather within the environment 100, movement of the animal 102, eating habits associated with the animal 102, frequency, spectral/cepstral cues, vocal quality/stability, prosody, temporal output, and amplitude stability, and/or motor processes. In an embodiment, the biomarkers 106 may be acoustic-based or non-acoustic based.

In an embodiment, the ML model(s) 136 may be used to process the audio data 112 and/or the sensor data 116 for determining the biomarkers 106. For example, the ML model(s) 136 may be previously trained to determine the biomarkers 106. In an embodiment, the ML model(s) 136 may receive the audio data 112 and/or the sensor data 116 as an input, and output an indication associated with whether the audio data 112 and/or the sensor data 116 are indicative of the biomarkers 106. In an embodiment, the ML model(s) 136 may output a score associated with the biomarkers 106, where the score may indicate likelihood or probability of the vocalizations and/or behavior of the animal 102 being associated with the biomarkers 106.

At 208, the process 200 may include determining whether the biomarker(s) satisfy predetermined threshold(s). For example, the biomarkers 106 may be compared to reference biomarker(s), or characteristic(s) that are indicative of the welfare, behavioral, and/or pathological states 108. Any number of the biomarkers 106 may be considered when determining whether the vocalizations and/or behavior are indicative of the welfare, behavioral, and/or pathological states 108. In an embodiment, the ML model(s) 136 may be used for determining whether the biomarkers 106 are indicative of satisfying threshold(s) associated with the vocalizations and/or behavior containing certain pathological states. As non-limiting examples, the biomarkers 106 may be associated with a pitch, amplitude, and/or tone associated with the vocalizations of the animal 102, and the pitch, amplitude, and/or tone may be compared to respective threshold(s). Based on such comparison, for example, the process 200 may determine whether the pitch, amplitude, tone, etc. are indicative of certain welfare, behavioral, and/or pathological states 108. Moreover, in an embodiment, audio fingerprint(s) 128 may be determined for comparison with one or more reference signatures to determine the welfare, behavioral, and/or pathological states 108 of the animal 102. If, at 208, the process 200 determines that the biomarkers 106 do not satisfy the threshold(s), the process 200 may follow the “NO” route and proceed to 210.

At 210, the process 200 may include causing output of one or more first notification(s). For example, the I/O component(s) 134 of the veterinary acoustic and biofeedback device 104 may output notification(s) 130, where the notification(s) 130 may indicate that the vocalizations and/or behavior is not associated with a particular welfare, behavioral, and/or pathological states 108. In an embodiment, the notification(s) 130 may be output at the veterinary acoustic and biofeedback device 104, or the mobile device 132 of the caregiver 110. From 210, the process 200 may proceed to 202, whereby the process 200 may continue to receive the audio data 112 and/or the sensor data 116 for determining the welfare, behavioral, and/or pathological states 108.

Returning to 208, if the process 200 determines that the biomarker(s) 106 satisfy the threshold(s), the process 200 may follow the “YES” route and proceed to 212. At 212, the process 200 may include causing output of one or more second notification(s). For example, the I/O component(s) 134 of the veterinary acoustic and biofeedback device 104 may output notification(s) 130, where the notification(s) 130 may indicate the welfare, behavioral, and/or pathological states 108.

At 214, the process 200 may include sending at least a portion of the audio data and/or the sensor data to a second device. For example, the veterinary acoustic and biofeedback device 104 may send the audio data 112 and/or the sensor data 116 to the mobile device 132 and/or the remote computing resource(s) 140. In an embodiment, the mobile device 132 and/or the remote computing resource(s) 140 may further process the audio data 112 and/or the sensor data 116, may store/log the audio data 112 and/or the sensor data 116 to maintain a history of the animal 102. In addition, the mobile device 132 may output the notification(s) 130 indicative of the welfare, behavioral, and/or pathological states 108. From 214, the process 200 may loop to 202, whereby the veterinary acoustic and biofeedback device 104 may continue to process and analyze vocalizations and/or behavior of the animal 102.

While various examples and embodiments are described individually herein, the examples and embodiments may be combined, rearranged, and modified to arrive at other variations within the scope of this disclosure.

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

What is claimed is:

1. A device to be worn by an animal, the device comprising:
one or more microphones;
an output component;
a network interface;
one or more processors; and
one or more non-transitory computer-readable media storing computer-executable instructions that, when executed by the one or more processors, cause the device to perform acts comprising:
receiving, from the one or more microphones, audio data associated with sound captured within an environment of the animal,
determining, based at least in part on the audio data, that the sound is associated with the animal,
determining, based at least in part on the sound being associated with the animal, one or more biomarkers associated with the sound,
determining a threshold associated with the animal having a behavioral or pathological state,
determining, based at least in part on the one or more biomarkers satisfying the threshold, that the sound is indicative of the animal having the behavioral or pathological state,
causing, based at least in part on the one or more biomarkers satisfying the threshold, output of a first notification on the output component, and
sending data associated with a second notification to be output on an electronic device associated with a caregiver of the animal.

2. The device of claim 1, further comprising a sensor, the acts further comprising receiving, from the sensor, sensor data, wherein:
determining that the sound is associated with the animal is based at least in part on the sensor data; and
determining the one or more biomarkers is based at least in part on the sensor data.

3. The device of claim 1, the acts further comprising:
receiving, from the one or more microphones, second audio data associated with a second sound captured within the environment;
determining, based at least in part on the second audio data, that the second sound is associated with the environment; and
determining one or more second biomarkers associated with the second sound,
wherein determining that the sound is indicative of the animal having the behavioral or pathological state is based at least in part on the one or more second biomarkers.

4. The device of claim 1, the acts further comprising:
determining an audio signature associated with the behavioral or pathological state pathological state;
determining, based at least in part on the one or more biomarkers, an audio signature associated with the sound; and
determining a similarity between the audio signature associated with the behavioral or pathological state and the audio signature associated with the sound,
wherein determining that the sound is indicative of the animal having the behavioral or pathological state is based at least in part on the similarity.

5. The device of claim 1, further comprising one or more attachment mechanisms for coupling the device to the animal.

6. The device of claim 1, wherein determining the one or more biomarkers is based at least in part on:
providing, as an input to a machine-learned (ML) model trained to identify the the behavioral or pathological state, the audio data; and
receiving, as an output from the ML model, an indication associated with the one or more biomarkers.

7. The device of claim 1, wherein the one or more biomarkers include at least one of a pitch of the sound, a tone associated with the sound, a pause in the sound, a duration of the sound, or an amplitude of the sound.

8. A device comprising:
one or more sensors;
one or more output components;
one or more processors; and
one or more non-transitory computer-readable media storing computer-executable instructions that, when executed by the one or more processors, cause the device to perform acts comprising:
receiving, from the one or more sensors, data,
determining that the data is indicative of sound generated from an animal,
determining, based at least in part on the data being indicative the sound generated from the animal, one or more biomarkers associated with the sound, the one or more biomarkers including at least an amplitude associated with the sound,
determining that the one or more biomarkers fail to satisfy a threshold associated with a pathological state of the animal, and
causing, based at least in part on the one or more biomarkers failing to satisfy the threshold, output of a notification via the one or more output components.

9. The device of claim 8, wherein the one or more sensors comprise at least one of:
at least one microphone; or
at least one of an internal measurement unit (IMU), an accelerometer, a gyroscope, or a piezoelectric sensor.

10. The device of claim 8, wherein the one or more output components comprise at least one of a lighting element or a speaker.

11. The device of claim 8, the acts further comprising sending a second notification to an electronic device.

12. The device of claim 8, wherein the device is configured to be worn by the animal.

13. The device of claim 8, the acts further comprising:
receiving, from the one or more sensors, second data;
determining that the second data is indicative of second sound generated from the animal;
determining, based at least in part on the second data being indicative the second sound generated from the animal, one or more second biomarkers associated with the second sound;
determining that the one or more second biomarkers satisfy the threshold, and
causing, based at least in part on the one or more second biomarkers satisfying the threshold, output of a third notification via the one or more output components.

14. The device of claim 8, wherein determining the one or more biomarkers is based at least in part on:
providing, as an input to a machine-learned (ML) model trained to identify the pathological state; and
receiving, as an output from the ML model, an indication associated with the one or more biomarkers.

15. The device of claim 8, wherein the one or more sensors comprise at least one of a microphone, an accelerometer, an inertial measurement unit (IMU), a GPS, or a temperature sensor.

16. A device comprising:
a first microphone;
a second microphone;
one or more output components;
one or more processors; and
one or more non-transitory computer-readable media storing computer-executable instructions that, when executed by the one or more processors, cause the device to perform acts comprising:
receiving, from the first microphone, first audio data associated with a sound captured in an environment,
receiving, from the second microphone, second audio data associated with the sound,
determining, based at least in part on the first audio data and the second audio data, that the sound is associated with an animal,
determining, based at least in part on the sound being associated with the animal, one or more characteristics associated with the sound,
determining, based at least in part on the one or more characteristics, that the sound is indicative of a pathological state, and
causing, based at least in part on the one or more characteristics being indicative of the pathological state, output of a notification via the one or more output components.

17. The device of claim 16, the acts further comprising sending data associated with the one or more characteristics to an electronic device.

18. The device of claim 16, further comprising one or more sensors that include at least one of an accelerometer, a gyroscope, an internal measurement unit (IMU), or a piezoelectric sensor, the acts further comprising receiving, from the one or more sensors, sensor data, wherein:
determining that the sound is associated with the animal is based at least in part on the sensor data; and
determining the one or more characteristics associated with the sound is based at least in part on the sensor data.

19. The device of claim 16, the acts further comprising determining one or more characteristics associated with an environment of the animal, and wherein determining that the sound is indicative of the pathological state is based at least in part on the one or more characteristics associated with the environment.

20. The device of claim 16, wherein determining the one or more characteristics is based at least in part on:
providing, as an input to a machine-learned (ML) model trained to identify the pathological state; and
receiving, as an output from the ML model, an indication associated with the one or more characteristics.
